(19) United States
(12) Patent Application Publication
(10) Pub. No.: US 2005/0148809 A1
(43) Pub. Date: Jul. 7, 2005

(54) High power therapeutic magnetic jewelry

(76) Inventor: Timothy P. Delaney, Portland, OR (US)

Correspondence Address: Donald W. Meeker;Patent Agent, 924 East Ocean Front #E, Newport Beach, CA, 92661, US

(21) Appl. No.: 10/748,004

(22) Filed: Dec. 29, 2003

(51) Int. Cl. A61B 17/52; A44C 5/00; A61N 1/00; A61N 2/00

(52) U.S. Cl. 600/15; 600/9; 63/3

(57) ABSTRACT

Very powerful magnetic links interconnected only by their magnetic attraction for each other form modular jewelry that can be placed at strategic points on the body for therapeutic purposes.

### BACKGROUND OF THE INVENTION

1. Field of the Invention

The present invention relates to jewelry used in magnetic therapy, and particularly to jewelry which uses very powerful extremely high gauss industrial magnets comprised of rare earth (neodymium) magnetic links interconnected only by their magnetic attraction for each other which connect in a circle to form jewelry with no cords or chains or anything interconnecting them except the magnetic attraction.

2. Description of the Prior Art

It is believed that magnetic energy works under the same principles as the ancient Chinese practice of acupuncture. Acupuncture, a traditional Chinese therapy, originated over three thousand years ago and is based on the belief that health and the body's overall well-being are determined by a balanced flow of “Chi”, the vital life energy present in all living organisms. According to the acupuncture theory, “Chi” circulates in the body along twelve major energy pathways called meridians, which are each linked to specific organs and body electrical systems. There is a specific relationship between acupuncture points, meridians and the electrical currents of the body. The electricity is generated by an interaction of positively and negatively charged ions. As long as the flow of energy remains unimpeded and in balance, it is believed that we remain physically and mentally balanced, and therefore in good health.

Proper use of magnets for pain has been proven to enhance the body's natural ability to heal itself, not just mask symptoms as many other treatments do. By using a properly designed north pole magnet on pain or illness, you can immediately start the healing process. It has been reported that biomagnets can be placed at strategic points on the body to positively influence pain control by directing magnetic fields to acupuncture points, acupuncture meridians, and the surrounding hard and soft tissue. For example, clothing that has discrete magnets attached thereto for producing healing effects at acupuncture points has been disclosed in prior art U.S. Pat. No. 5,720,046, which issued Feb. 24, 1998 to Lopez et al. Lopez discloses clothing and other personal wear which utilize discrete magnets to direct magnetic fields towards acupuncture points along the human torso, acupuncture points on the head along the temporal region, soft spot and upper most vertebrae region at the base of the skull, and acupuncture points on the body appendages.

Prior art U.S. Pat. No. 6,427,486, issued Aug. 6, 2002 to Yellen, illustrates a pinless articulated band that is formed of a lineal array of metallic elements arranged in a repeating pattern. The elements are separably bonded to one another through magnetic forces acting therebetween. The band is characterized in that it is free of pins and other mechanical structural agents for interconnecting the band elements to one another. The configuration of the elements themselves is such that each has at least two surfaces, and such that these surfaces include one that is curved or arcuate. In a preferred embodiment of the invention, the elements include cylinders and discs that are of the same diameters. The cylinders and discs are so arranged that their axes are parallel to one another. In a closed loop configuration, the band exhibits aesthetic design features and finds use as a bracelet. The Yellen magnets are less powerful than those of the present invention and seem to require overlapping in order to stay together. There does not appear to be a healing intention for the Yellen patent.

Prior art U.S. Pat. No. 6,544,164, issued Apr. 8, 2003 to Fan, indicates a light wave energy health magnetic ring, which includes a plurality of blocks of different shapes formed from superconductive magnetic materials, natural crystals containing strong natural magnetic fields, and infrared ceramic beads. Bottom portions of the superconductive magnetic materials are formed with recesses for embedding crude crystals therein. Round, flat and circular-shaped, or wheel-shaped natural crystals and ceramic beads are spacedly disposed between adjacent superconductive magnetic materials such that the natural crystals are arranged in diagonally opposite positions. These elements are connected in a ring shape for wearing by a user. The magnetic waves can penetrate the muscles and bones of the body of the user to magnetize the cells of the body to thereby improve the circulation functions of the body.

Prior art U.S. Pat. No. 658,027, issued Sep. 18, 1900 to Steiger, indicates a therapeutic device that comprises magnetic disks provided with slots, several of which may be threaded on a tape for use in treatment. Alternately, the disks may be used individually.

Prior art U.S. Pat. No. 5,806,346, issued Sep. 15, 1998 to Schlinger, provides a magnetic pendant necklace set method of manufacture. The method of inexpensively making a magnetic pendant necklace set relies upon affixing opposite-polarity magnets of at least 27 megagauss oersteds in pole strength to each of two, substantially-flat ornamental settings with protruding rings to which the opposite ends of any type of necklace can be attached. The ornamental settings function as a pendant clasp when joined together by their magnet centers and also as a secure mount for the easy attachment of magnet-studded, pendant ornaments.

Prior art U.S. Pat. No. 5,195,335, issued Mar. 23, 1993 to Hart, concerns a necklace with a flexible string that passes through a plurality of bead-like members, some of the beads on the string being magnetized so that when two of such beads are placed in the juxtaposed position, the necklace assumes a unique shape.

Prior art U.S. Pat. No. 4,912,944, issued Apr. 3, 1990 to Crosley, provides an article of jewelry that comprises a base element, which supports an ornamented substrate element thereon by magnetic attraction. Both the base element and the substrate element have magnetically attractable properties and at least one of them has the properties of a permanent magnet, whereby the substrate element is interchangeable with other like elements bearing different ornamentation.

Prior art U.S. Pat. No. 6,594,871, issued Jul. 22, 2003 to Hoffman, illustrates a jewelry element that includes a magnetic arrangement for ready substitution of multiple decorative elements. The element, which may be incorporated into a number of common jewelry pieces including a cuff link, earring, a stud for a tuxedo shirt, a ring or a pendant, includes a frame having a recessed central portion for receiving a decorative element. A pair of magnets is press-fit within a circular slot at the bottom of the recess and another magnet is adhesively fixed to the bottom of the decorative element. An aperture in the bottom of the recessed portion of the frame provides access for a stylus for selectively dislodging the decorative element.

Prior art U.S. Pat. No. 6,591,462, issued Jul. 15, 2003 to Fuhrman, is for a magnetic jewelry clasp that has two bodies and a safety catch. Each body has a surface magnetically attracted to the other. The clasp has a safety catch that includes an arm hinged to one of the bodies. The arm may be swung about the hinge between an open position and a closed position. A further magnetic attraction keeps the arm in the closed position until opened with a fingernail. A retaining wall may extend generally perpendicular to a direction of elongation of the arm to block a path that the other of the bodies could travel if the bodies were to separate from each other while the safety catch is in the closed position. If desired, a male connector may be inserted into a female connector to prevent separation of the bodies from each other. The male connector and female connector are usable in tandem with the retaining wall or in lieu thereof.

Prior art U.S. Pat. No. 5,732,451, issued Mar. 31, 1998 to Mars, concerns a device for magnetically attaching jewelry, name tags or the like to garments. The device includes inner and outer magnets and an anchor attached to the outer magnet. A pin-secured jewelry article or name tag is secured to the garment by passing the pin through a passageway in the anchor and then bringing the inner and outer magnets into magnetically close proximity on opposite sides of the garment.

What is needed is powerful industrial magnets which hold together side-by-side or end-to-end without the need for overlapping the magnets, which powerful magnets may be assembled without other interconnecting means into a variety of different types of jewelry, and which magnets provide therapeutic as well as aesthetic value.

### SUMMARY OF THE INVENTION

An object of the present invention is to provide powerful industrial extremely high gauss (starting at but not limited to 6,000 gauss) magnets in a variety of shapes and sizes which hold together side-by-side or end-to-end without the need for overlapping the magnets, which powerful magnets may be assembled in circles without other interconnecting means into a variety of different types of jewelry, including bracelets, necklaces, anklets, and rings, and which magnets provide therapeutic as well as aesthetic value.

Another object of the present invention is to provide a core magnet that is made up of sintered ceramic, which is then formed and coated with nickel (with the exception of the epoxy) and then finished with any of a variety of finishes including silver, nickel, black epoxy, 24k electro-plated gold, platinum, and black nickel for a wide variety of decorative colors and finish looks which can all be worn together in different combinations

One more object of the present invention is to provide components that are formed in any of a variety of shapes including cubes, cylinders/rods, spheres, blocks, and squares to greatly vary the ornamental choices for assembling the magnets into jewelry of similar and mixed shapes.

An additional object of the present invention is to provide modular jewelry that is held together by magnetic attractive force only, requiring no fasteners of any kind, such as clasps which are difficult to operate at times.

A related object of the present invention is to provide individual magnets which may be customized according to the size to fit different needs.

A further object of the present invention is to provide a modular high-powered magnetic jewelry system with vertical as well as horizontal magnetic fields, allowing the wearer to interchange shapes and sizes.

In brief, a modular magnetic jewelry system in which each individual magnetic link in the jewelry is diametrically magnetized, utilizing a north/south relationship. The north field has to connect to the south field to link together properly. The links are powerful industrial extremely high gauss magnets which hold together side-by-side or end-to-end without the need for overlapping the magnets. The powerful magnets may be assembled in circles without other interconnecting means into a variety of different types of jewelry, including bracelets, necklaces, anklets, and rings. The modular jewelry is held together by magnetic attractive force only, requiring no fasteners of any kind. Each link is a core magnet that is made up of sintered ceramic, which is then formed and coated with nickel (with the exception of the epoxy) and then finished with any of a variety of finishes including silver, nickel, black epoxy, 24k electro-plated gold, platinum, and black nickel for a wide variety of decorative colors and finish looks which can all be worn together in different combinations. The links are formed in any of a variety of shapes including cubes, cylinders/rods, spheres, blocks, and squares to greatly vary the ornamental choices for assembling the magnets into jewelry of similar and mixed shapes. The links are provided with vertical as well as horizontal magnetic fields, allowing the wearer to interchange shapes and sizes. Any of the wide variety of shapes, colors, sizes and textures can be linked together to form the jewelry.

The modular magnetic jewelry creates a therapeutic magnetic field around a portion of a user wearing the jewelry and enhances the body's natural ability to heal itself. The high gauss magnets can be placed at strategic points on the body to positively influence pain control by directing magnetic fields to acupuncture points, acupuncture meridians, and the surrounding hard and soft tissue.

An advantage of the present invention is that the wearer may create their jewelry design.

Another advantage of the present invention is that the design may be changed at any time.

An additional advantage of the present invention is that it does not require fastening devices.

One more advantage of the present invention is that the magnets provide therapeutic benefits.

Yet another advantage of the present invention is that the magnets are formed a wide variety of shapes, colors, sizes and textures.

One more advantage of the present invention is that the magnets may be customized according to the size to fit different needs

Still another advantage of the present invention is that the links will stay in place.

A further advantage of the present invention is that the links may be assembled on various parts of the body.

### BRIEF DESCRIPTION OF THE DRAWINGS

These and other details of my invention will be described in connection with the accompanying drawings, which are furnished only by way of illustration and not in limitation of the invention, and in which drawings:

FIG. 1 is a perspective view of two pieces of magnetic jewelry using spaced apart rectangular block magnets and a series of cylindrical magnets in between all interconnected side by side to form a circular bracelet shown on a wrist of a user and a circular ring shown on a finger of the wearer;

FIG. 2 is a perspective view of a series of cylindrical magnets with different magnetic pole orientations so that cylindrical magnets with a transverse magnetic field as shown in FIG. 3D are spaced apart in vertical orientations with pairs of cylindrical magnets each with an axial magnetic field as shown in FIG. 3E are linked horizontally between the vertical cylinders;

FIG. 3A is a perspective view of a magnetic cube;

FIG. 3B is a perspective view of a magnetic rectangular block;

FIG. 3C is a perspective view of a magnetic sphere;

FIG. 3D is a perspective view of a magnetic cylinder with a transverse magnetic field;

FIG. 3E is a perspective view of a magnetic cylinder with an axial magnetic field;

FIG. 4 is a plan view of a circular magnetic jewelry configuration formed of alternating magnetic cubes and spheres, shown around a neck of a wearer;

FIG. 5 is a side elevational view of the circular magnetic jewelry configuration of FIG. 4 worn as an anklet.

### BEST MODE FOR CARRYING OUT THE INVENTION

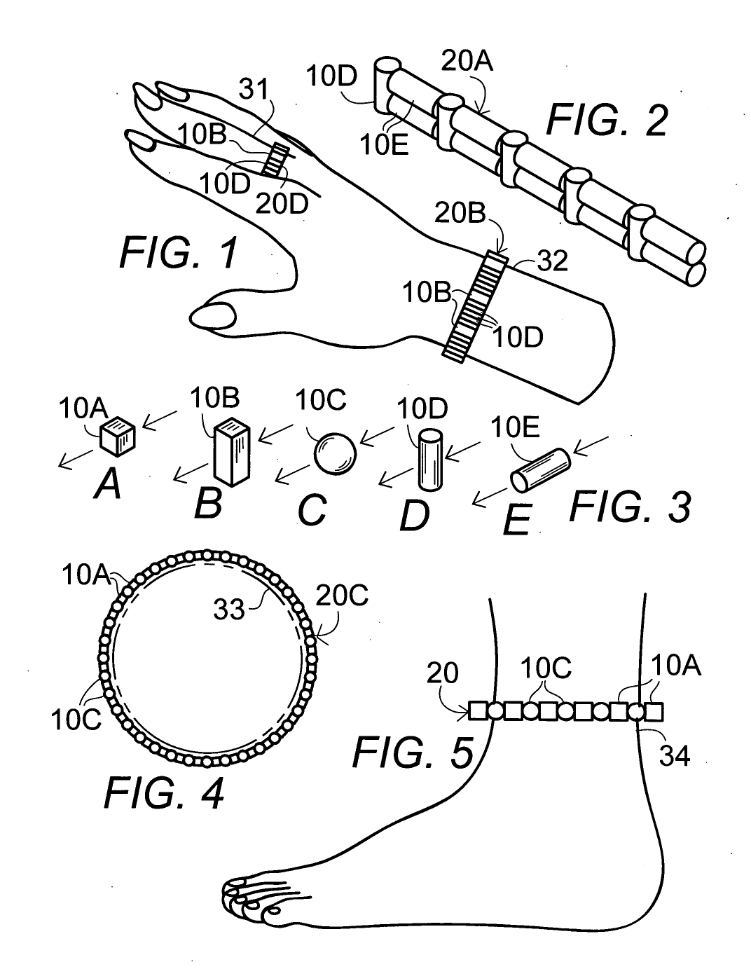

In FIGS. 1-5, a magnetic jewelry system 20 and 20A-20D of magnets 10A-10E linked together by magnetic attraction in decorative therapeutic circular configurations worn as jewelry 20 and 20A-20D, which comprises a series of magnets 10A-10E each having a magnetic force of 6,000 gauss or more. Each of the magnets 10A-10E comprises a core of sintered ceramic having a coating of metal and a form and an exterior finish adapted for use in magnetic jewelry 20 and 20A-20D. The finish may comprise finishes including silver, nickel, black epoxy, 24k electro-plated gold, platinum, or black nickel which may be intermixed in a piece of magnetic jewelry 20 and 20A-20D for a wide variety of finish looks which can be worn together in different combinations.

In FIGS. 3A-3E, each of the magnets 10A-10E is diametrically magnetized utilizing a north/south relationship with the arrow facing the north magnetic direction in each piece. The north field of each of the magnets 10A-10E connects to a south field of an adjacent magnet 10A-10E to link together with each of the magnets 10A-10E forming an individual magnetic link 10A-10E in a circular configuration, as seen in FIG. 4 (showing a circular piece of jewelry 20C worn as a necklace around a neck 33 of a wearer), which is worn as a piece of magnetic jewelry 20 and 20A-20D, as seen in FIGS. 1, 4, and 5. The magnetic attraction between each of the adjacent magnets 10A-10E is the only interconnecting means of the piece of magnetic jewelry 20 and 20A-20D. Each of the magnets 10A-10E may have a shape consisting of a cube 10A, a cylinder or rod 10D and 10E, a sphere 10C, or a rectangular block 10B. The shapes 10A-10E can be used together in a single piece of magnetic jewelry 20 and 20A-20D for a variety of decorative choices.

The piece of magnetic jewelry 20 and 20A-20D creates a therapeutic magnetic field around a portion of a user wearing the piece of magnetic jewelry 20 and 20A-20D. The piece of magnetic jewelry 20 and 20A-20D may be assembled into a circular configuration and worn as a bracelet 20B shown in FIG. 1, a necklace 20C shown in FIG. 4, an anklet 20 shown in FIG. 5, or a ring 20D shown in FIG. 1.

In practice, a variety of different types of therapeutic magnetic jewelry 20 and 20A-20D, including bracelets 20B seen in FIG. 1, necklaces seen in FIG. 4, anklets 20 seen in FIG. 5 and rings seen in FIG. 1 may be made from the individual magnetic links 10A-10E. The user assembling the jewelry 20 and 20A-20D measures the body part on which the magnetic jewelry 20 and 20A-20D is to be worn. The user would choose the shape, color, and texture of individual magnetic links 10A-10E, which they prefer. The shapes, which may be a cube 10A, a cylinder or rod 10D and 10E, a sphere 10C, or a rectangular block 10B, can be used together in a single piece of magnetic jewelry 20 and 20A-20D for a variety of decorative choices, as shown in FIGS. 1, 2, 4 and 5. The finish may be chosen from silver, nickel, black epoxy, 24k electro-plated gold, platinum, or black nickel which may be intermixed in a piece of magnetic jewelry 20 and 20A-20D for a wide variety of finish looks which can be worn together in different combinations. The individual magnets 10A-10E are assembled on a flat surface to the length required, for example 7″ long for a bracelet 20B. The whole magnetic piece of jewelry 20 and 20A-20D is lifted from the flat surface then wrapped around the body part, which it was created for. The north field of one end magnet 10A-10E is then connected to a south field of the other end magnet 10A-10E to form a circular configuration around the body part, as seen in FIGS. 1, 4, and 5.

The magnetic piece of jewelry 20 and 20A-20D may be worn to create a therapeutic magnetic field around a portion of a user wearing the jewelry 20 and 20A-20D and enhance the body's natural ability to heal itself. The jewelry 20 and 20A-20D assembled of high gauss magnets 10A-10E can be placed at strategic points on the body to positively influence pain control by directing magnetic fields to acupuncture points, acupuncture meridians, and the surrounding hard and soft tissue.

The individual magnetic links 10A-10E may be sold to the consumer by the inch or in a kit consisting of a preset number of magnetic links 10A-10E. The consumer may then arrange the magnetic links 10A-10E to form a piece of magnetic jewelry 20 and 20A-20D that they require.

It is understood that the preceding description is given merely by way of illustration and not in limitation of the invention and that various modifications may be made thereto without departing from the spirit of the invention as claimed.

1. A magnetic jewelry system of magnets linked together by magnetic attraction in decorative therapeutic circular configurations worn as jewelry, the system comprising:
a series of magnets each having a magnetic force of at least 6,000 gauss and each of the magnets comprising a core of sintered ceramic having a coating of metal and having a form and an exterior finish adapted for use in magnetic jewelry, each of the magnets diametrically magnetized utilizing a north/south relationship with each of the magnets having a north field connected to a south field of an adjacent one of the magnets to link together with each of the magnets forming an individual magnetic link in a circular configuration adapted to be worn as a piece of magnetic jewelry having a magnetic attraction between each of the adjacent magnets as the only interconnecting means;
wherein the piece of magnetic jewelry creates a therapeutic magnetic field around a portion of a user wearing the piece of magnetic jewelry.

2. The system of claim 1 wherein each of the magnets has a shape selected from the list of shapes consisting of a cube, a cylinder, a rod, a sphere, and a rectangular block, and the shapes can be used together in a single piece of magnetic jewelry for a variety of decorative choices.

3. The system of claim 1 wherein the piece of magnetic jewelry is adapted for use as one of a list of pieces of jewelry consisting of a bracelet, a necklace, an anklet, and a ring.

4. The system of claim 1 wherein the finish comprises any of a variety of finishes selected from the list of finishes including silver, nickel, black epoxy, 24k electroplated gold, platinum, and black nickel which may be intermixed in a piece of magnetic jewelry for a wide variety of finish looks which can be worn together in different combinations.
